SEQUENCE LISTING (1 sequence)

SEQ ID NO: 1 - protein, 224 aa, Triticum aestivum
MGYSKTLVAGLFAMLLLAPAVLATDPDPLQDFCVADLDGKAVSVNGHTCKPMSEAGDDFLFSSKLAKAGNTSTPNGSAVTELDVAEWPGTNTLGVSMNRVDFAPGGTNPPHIHPRATEIGIVMKGELLVGILGSLDSGNKLYSRVVRAGETFLIPRGLMHFQFNVGKTEASMVVSFNSQNPGIVFVPLTLFGSNPPIPTPVLTKALRVEARVVELLKSKFAAGF

(19) United States
(12) Patent Application Publication
(10) Pub. No.: US 2023/0321202 A1
(43) Pub. Date: Oct. 12, 2023

(54) AMERICAN CHESTNUT LEAF COMPOSITIONS COMPRISING OXALATE OXIDASE AND METHODS FOR TREATMENT OF OXALATE-RELATED DISORDERS

(71) Applicants: UNIVERSITY OF FLORIDA RESEARCH FOUNDATION, INCORPORATED, Gainesville, FL (US); The Research Foundation for The State University of New York (RFSUNY), Albany, NY (US)

(72) Inventors: Paul R. Dominguez-Gutierrez, Archer, FL (US); William L. Donelan, Gainesville, FL (US); Tania Quesada, Archer, FL (US); William A. Powell, Syracuse, NY (US)

(73) Assignees: UNIVERSITY OF FLORIDA RESEARCH FOUNDATION, INCORPORATED, Gainesville, FL (US); The Research Foundation for The State University of New York (RFSUNY), Albany, NY (US)

(21) Appl. No.: 18/185,315

(22) Filed: Mar. 16, 2023

Related U.S. Application Data
(60) Provisional application No. 63/320,627, filed on Mar. 16, 2022.

(51) Int. Cl. A61K 38/44; A61K 36/49; A61K 47/46; C07C 51/43; A61K 9/00; C12N 9/02; A23F 3/14; A23F 3/16

(52) U.S. Cl. CPC A61K 38/44; A61K 36/49; A61K 47/46; C07C 51/43; A61K 9/0095; C12N 9/0008; C12Y 102/03004; A23F 3/14; A23F 3/166; A61K 2236/331; A61K 2236/15; A61K 2236/37

(57) ABSTRACT

The invention provides American chestnut leaf compositions comprising oxalate oxidase. The compositions reduce oxalate levels in dietary sources of oxalate and are useful in reducing oxalate levels and reducing stone disease in patients. The invention provides methods of extracting and purifying oxalate oxidase from American chestnut leaf. The invention provides methods of extracting oxalate from biological samples.

### CROSS REFERENCE TO RELATED APPLICATION

This application claims the benefit of U.S. 63/320,627, filed Mar. 16, 2022, which is incorporated by reference in its entirety for all purposes.

### REFERENCE TO A SEQUENCE LISTING

The Sequence Listing written in file 591832SEQLST.xml is 2,034 bytes, was created on May 31, 2023, and is hereby incorporated by reference.

### BACKGROUND OF THE INVENTION

The American chestnut tree (ACT, Castanea dentata) was once considered one of the most important North American forest trees due to its expansive range and nut products. The tea from its leaves was even used as a popular home remedy for cough/colds and was commercially available in the 1800s. ACT has become functionally extinct due to “chestnut blight” caused by Cryphonectria parasitica. This fungus, an accidental import from Asia in the early 1900s, secretes oxalate at lethal levels and accelerates tissue decay on tree hosts, like the ACT, that do not have oxalate degrading enzymes. Beginning in 1990, through efforts of the American Chestnut Research & Restoration Program at the State University of New York (SUNY), College of Environmental Science and Forestry, a genetically modified ACT (GM-ACT) was developed containing a wheat gene that codes for the enzyme oxalate oxidase (OxOx) and expresses it at high levels, making the genetically modified ACT tolerant to blight.

Darling 58 is a transgenic American chestnut tree (Castanea dentata) overexpressing wheat (Triticum aestivum) oxalate oxidase to resist chestnut blight caused by Cryphonectria parasitica. It was created by the American Chestnut Research & Restoration Program at the State University of New York (SUNY) College of Environmental Science and Forestry by the Laboratory of Dr. William Powell. According to researcher's EPA petition for determination of nonregulated status for blight-tolerant Darling 58 American chestnut, the “chestnuts retain 100 percent of their natural complement of genes; no native genes or alleles have been removed or replaced, and expression of nearby genes is not affected.” Therefore, the trees are genetically identical to the native American chestnuts except for the blight-resistant trait. There are no negative human health issues regarding consumption of oxalate oxidase since it is present in many food crops (Newhouse, A., (2020_FinalChestnut Winter2020, Safety tests on transgenic American). Humans have been eating oxalate oxidase in bread for millennia.

Kidney stones, primarily containing the mineral calcium oxalate, affect one in ten Americans and cost over 10 billion dollars annually to treat. Many stone formers excrete excessive amounts of oxalate in their urine due to the over-ingestion of oxalate from dietary sources. Once a person has a stone, 50 to 75% of individuals will have another stone between 5 to 10 years. This diagnosis is typically made by testing 24 hours of urine using the OxOx enzyme extracted from 10-day old barley seedlings at yields of 1 unit/100 gm (Trinity Biotech Oxalate Kit, Cat. 591D, Bray, Co. Wicklow, Ireland)).

Tea, an aromatic beverage prepared from leaves of the Camellia sinensis shrub, is high in soluble oxalate, and its intake is generally discouraged in stone formers. It is useful in the art to have additional sources of oxalate oxidase enzyme and to have improved methods of reducing oxalate in tea. GM-ACT leaves are useful as a therapeutic in reducing urinary oxalate levels in tea drinkers who are at high stone risk.

### BRIEF SUMMARY OF THE CLAIMED INVENTION

In one aspect, the invention provides a composition comprising tea leaves and leaves of American chestnut tree plants expressing wheat oxalate oxidase. In some compositions, the leaves of American chestnut tree plants expressing wheat oxalate oxidase are ground.

In another aspect, the invention provides a method for producing a brewed tea with reduced oxalate levels, comprising: (a) combining tea leaves with leaves of American chestnut tree plants expressing wheat oxalate oxidase; (b) steeping the tea leaves and leaves of American chestnut tree plants expressing wheat oxalate oxidase in boiling water to brew the tea; and (c) collecting the brewed tea, wherein the brewed tea has reduced oxalate levels compared to tea brewed without leaves of American chestnut tree plants expressing wheat oxalate oxidase. In some methods, the leaves of American chestnut tree plants expressing wheat oxalate oxidase are ground.

In another aspect, the invention provides a method of reducing stone formation in a patient, comprising administering a therapeutically effective amount of leaves of American chestnut tree plants expressing wheat oxalate oxidase to the patient, wherein stone formation is reduced in the patient.

In another aspect, the invention provides a method of reducing urinary oxalate levels in a patient, comprising administering a therapeutically effective amount of leaves of American chestnut tree plants expressing wheat oxalate oxidase to the patient, wherein urinary oxalate levels are reduced in the patient.

In another aspect, the invention provides a method for purifying oxalate oxidase from leaves of American chestnut tree plants expressing wheat oxalate oxidase, the method comprising: preparing an extract from the leaves, wherein the extract is prepared in the presence of 0.1% sodium dodecyl sulfate at 60° C., precipitating proteins in said extract by bringing said extract to at least about 30% (w/v) saturation with ammonium sulfate, removing said precipitated proteins and bringing said extract to at least about 60% (w/v) saturation with ammonium sulfate to precipitate said oxalate oxidase present in said extract and recovering said oxalate oxidase.

In another aspect, the invention provides an oxalate oxidase composition having a specific activity of at least about 9 enzyme units per 100 g of starting leaves of American chestnut tree plants expressing wheat oxalate oxidase after extraction in the presence of 0.1% sodium dodecyl sulfate at 60° C. and ammonium sulfate precipitation. In some oxalate oxidase compositions, the oxalate oxidase is produced according to a process comprising the steps of preparing an extract from leaves of American chestnut tree plants expressing wheat oxalate oxidase, wherein the extract is prepared in the presence of 0.1% sodium dodecyl sulfate at 60° C., precipitating proteins in said extract by bringing said extract to at least about 30% saturation (w/v) with ammonium sulfate, removing said precipitated proteins and bringing said extract to at least about 60% (w/v) saturation with ammonium sulfate to precipitate said oxalate oxidase present in said extract and recovering said oxalate oxidase.

In another aspect, the invention provides a method of reducing oxalate levels in a material, comprising adding an effective amount of any of the oxalate oxidase compositions herein to the material, wherein the oxalate level of the material is reduced. In some methods, the material is a food or beverage. In some methods, the food or beverage is selected from the group consisting of beer, spinach, rhubarb, okra, peanut butter, taro, bran flakes, soy, tofu, soymilk, nuts, almonds, cashews, peanuts, potatoes, beets, navy beans, raspberries, pumpkin, chocolate, swiss chard, eggplants, yams, avocados, tomato sauce, dates, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress. In some methods, the material is wood.

In another aspect, the invention provides a method for extracting oxalate from a biological sample, comprising: (a) solubilizing oxalate in the biological sample with 5 M HCl at 60° C. for 4 hours; (b) separating the solubilized oxalate from insoluble material; (c) precipitating oxalate from the solubilized oxalate with 12 M ammonium hydroxide; (d) washing the precipitated oxalate with 5 M ammonium hydroxide; and (e) resuspending the washed precipitated oxalate in EDTA at a concentration of 0.5 M or 1.0 M. In some methods, the biological sample is tea leaves, leaves of American chestnut tree plants expressing wheat oxalate oxidase, seagrass, spinach, or kidney stone. In some methods, the kidney stone is from a human.

### BRIEF DESCRIPTION OF THE DRAWINGS

FIG. 1 depicts oxalate levels in seagrass.

FIGS. 2A and 2B depict oxalate recovery of 5 mg CaOx spike. FIG. 2A depicts oxalate recovery from 5 mg spike, and FIG. 2B depicts recovery efficiency.

FIGS. 3A and 3B depict oxalate oxidase activity. FIG. 3A a comparison of GM-ACT leaf extract to 1/10 dilution of oxalate oxidase enzyme from the Trinity Biotech oxalate assay kit (Cat. 591D). FIG. 3B is a hydrogen peroxide (byproduct of oxalate degradation) standard curve for determination of activity.

FIG. 4 depicts results of an assay showing that GM-ACT leaves rapidly degrade oxalate.

FIG. 5 depicts soluble oxalate per gram of tea steeped with or without GM-ACT leaves.

FIG. 6 depicts soluble oxalate per serving of tea with or without GM-ACT leaves.

### DEFINITIONS

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

An individual is at increased risk of a disease if the subject has at least one known risk-factor (e.g., genetic, biochemical, family history, and situational exposure) placing individuals with that risk factor at a statistically significant greater risk of developing the disease than individuals without the risk factor.

The term “biological sample” refers to a sample of biological material within or obtainable from a biological source, for example a human or mammalian subject, or from a plant. Such samples can be organs, organelles, tissues, sections of tissues, bodily fluids, peripheral blood, blood plasma, blood serum, cells, molecules such as proteins and peptides, and any parts or combinations derived therefrom. The biological sample can be urine or kidney stone from a human or mammalian subject. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, as perceived by the subject. A “sign” refers to objective evidence of a disease as observed by a physician.

As used herein, oxalate oxidase (OxOx) refers to an oxalate:oxygen oxidoreductase enzyme. Oxalate oxidases are a group of well-defined enzymes capable of catalyzing the molecular oxygen (O₂)-dependent oxidation of oxalate to carbon dioxide and hydrogen peroxide according to the following reaction.

oxalate+O₂+2H⁺ [symbol] 2CO₂+H₂O₂

Isoforms of oxalate oxidase, and glycoforms of those isoforms, are included within this definition. OxOx from plants, bacteria and fungi are encompassed by the term, including the true cereal OXOs, such as wheat, barley, maize, oat, rice, and rye. Optionally, the OxOx will additionally be capable of superoxide dismutase activity, such as barley OxOx. In certain circumstances, OxOx is a soluble hexameric protein, including a trimer of OxOx glycoprotein dimers.

The term “individual” or “subject” refers to any mammal, including any animal classified as such, including humans, non-human primates, primates, baboons, chimpanzees, monkeys, rodents (e.g., mice, rats), rabbits, cats, dogs, horses, cows, sheep, goats, pigs, etc.

As used herein, “oxalate-related disorder” refers to a disease or disorder associated with pathologic levels of oxalic acid or oxalate, including, but not limited to stone disease, hyperoxaluria, primarily hyperoxaluria, enteric hyperoxaluria, idiopathic hyperoxaluria, ethylene glycol (oxalate) poisoning, idiopathic urinary stone disease, renal failure (including progressive, chronic, or end-stage renal failure), steatorrhea, malabsorption, ileal disease, vulvodynia, cardiac conductance disorders, inflammatory bowel disease, cystic fibrosis, exocrine pancreatic insufficiency, Crohn's disease, ulcerative colitis, nephrocalcinosis, urolithiasis, breast cancer, nephrolithiasis, secondary hyperoxaluria (SH), Zellweger spectrum disorders (ZSD), autism, oxalosis associated with end-stage renal disease, colitis, sarcoidosis, asthma, COPD, fibromyalgia, Zellweger syndrome, bariatric surgery and other enteric disease states. Such conditions and disorders may optionally be acute or chronic. Oxalate-related disorders associated with kidneys, bone, liver, gastrointestinal tract, and pancreas are well known. Further, it is well known that calcium oxalate can deposit in a wide variety of tissues including, but not limited to the eyes, blood vessels, joints, bones, muscles, heart, and other major organs leading to a number of oxalate-related disorders.

“Oxalic acid” exists predominantly in its salt form, oxalate (as salts of the corresponding conjugate base), at the pH of urine and intestinal fluid (pKₐ₁=1.23, pKₐ₂=4.19). Earnest, Adv. Internal Medicine 24:407-427 (1979). The terms “oxalic acid” and “oxalate” are used interchangeably throughout this disclosure. Oxalate salts comprising lithium, sodium, potassium, and iron (II) are soluble, but calcium oxalate is very poorly soluble in water, dissolving only to 0.58 mg/100 ml at 18° C. Earnest, Adv. Internal Medicine 24:407-427 (1979). Oxalic acid from food is also referred to as dietary oxalate. Oxalate that is produced by metabolic processes is referred to as endogenous oxalate. Circulating oxalate is the oxalate present in a circulating body fluid, such as blood.

The terms “therapeutically effective dose,” or “therapeutically effective amount,” refer to that amount of a compound that results in prevention, delay of onset of symptoms, or amelioration of symptoms of an oxalate-related condition, including hyperoxaluria, such as primary hyperoxaluria or enteric hyperoxaluria. A therapeutically effective amount will, for example, be sufficient to treat, prevent, reduce the severity, delay the onset, or reduce the risk of occurrence of one or more symptoms of a disorder associated with elevated oxalate concentrations. The effective amount can be determined by methods well known in the art and as described in subsequent sections of this description.

The terms “treatment,” “therapeutic method,” and their cognates refer to treatment and prophylactic/preventative measures. Those in need of treatment may include individuals already having a particular medical disorder as well as those who may ultimately acquire the disorder. The need for treatment is assessed, for example, by the presence of one or more risk factors associated with the development of a disorder, the presence or progression of a disorder, or likely receptiveness to treatment of a subject having the disorder. Treatment may include slowing or reversing the progression of a disorder.

Compositions or methods “comprising” or “including” one or more recited elements may include other elements not specifically recited. For example, a composition that “comprises” or “includes” an antibody may contain the antibody alone or in combination with other ingredients. When the disclosure refers to a feature comprising specified elements, the disclosure should alternatively be understood as referring to the feature consisting essentially of or consisting of the specified elements. Moreover, elements that are shown or described as being combined with other elements, can, in various embodiments, exist as stand-alone elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value.

Statistical significance means p≤0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

The present invention provides methods of purifying oxalate oxidase from GM-ACT leaves at high yield. The purified oxalate oxidase is useful in detecting oxalate in samples, for example biological samples from a human, an animal, or a plant. The purified oxalate oxidase is useful in a diagnostic test that measures oxalate in a biological sample.

The present invention provides methods of reducing oxalate levels in a food or beverage by combining the food or beverage with GM-ACT leaves. In some methods, GM-ACT leaves and tea leaves are brewed to produce a beverage for human consumption. The compositions and methods of the invention are useful in preventing and treating oxalate-related disorders in humans and animals.

Oxalate Oxidase

Oxalate oxidase (OxOx, EC 1.2.3.4) is expressed in higher plants and catalyzes the oxygen-dependent oxidation of oxalate to CO₂ with concomitant formation of H₂O₂. This reaction forms the basis of current assays for the detection of urinary oxalate levels (see, e.g., Trinity Biotech Oxalate Kit, Cat. 591D, Bray, Co. Wicklow, Ireland). OxOx is present in wheat, barley, beetroot stem and root, amaranthus leaves, sorghum and many other grains. In compositions herein, wheat OxOx is transgenically expressed in American Chestnut Tree (Zhang, B. et al., (2013) A threshold level of oxalate oxidase transgene expression reduces Cryphonectria parasitica-induced necrosis in a transgenic American chestnut (Castanea dentata) leaf bioassay, Transgenic Res. 2:973-982). OxOx is tightly bound in the cell wall as a mono-hexamer. The hexamer is what is found in nature; however, extracts and commercially available OxOx (for example that in Trinity Biotech Oxalate Assay) are monomers. Methods of the invention purify OxOx from GM-ACT as a hexamer. Having the purified OxOx in hexamer form provides improved activity over monomer form OxOx. At most, the monomer in solution can be 3 units per ml (Trinity Biotech assay) before it precipitates and becomes inactive. The invention provides GM-ACT powder with over 300 units per gram. The GM-ACT extract only has the equivalent of 0.1 units per gram of GM-ACT leaves and 10-day old barley seedlings (the commercial standard) have around 0.01 units per gram. A unit of OxOx activity is defined as “micromoles of substrate degraded per minute.”

Genetics of Oxalate Oxidase

The wheat oxalate oxidase sequence GF-2.8 was reported by Dr. Byron Lane:
Dratewka-Kos, E., Rahman, S., Grzelczak, Z. F., Kennedy, T. D., Murray, R. K., and Lane, B. G. (1989). Polypeptide structure of germin as deduced from cDNA sequencing. J. Biol. Chem. 264, 4896-4900.
>T. aestivum Oxalate Oxidase amino acid sequence (NCBI Reference Sequence:

XP_044377513.1)
(SEQ ID NO: 1)
MGYSKTLVAGLFAMLLLAPAVLATDPDPLQDFCVADLDGKAVSVNGHT
CKPMSEAGDDFLFSSKLAKAGNTSTPNGSAVTELDVAEWPGTNTLGVS
MNRVDFAPGGTNPPHIHPRATEIGIVMKGELLVGILGSLDSGNKLYSR
VVRAGETFLIPRGLMHFQFNVGKTEASMVVSFNSQNPGIVFVPLTLFG
SNPPIPTPVLTKALRVEARVVELLKSKFAAGF

Compositions

GM-ACT leaves of the invention may be added to foods or beverages. Exemplary foods or beverages to which GM-ACT leaves may be added include for example, beer, spinach, rhubarb, okra, peanut butter, taro, bran flakes, soy, tofu, soymilk, nuts (for example almonds, cashews, peanut), potatoes (including potato chips and French fries), beets, navy beans, raspberries, pumpkin, chocolate, swiss chard, eggplants, yams, avocados, tomato sauce, dates, and those reported at www.kidneystones.uchicago.edu./how-to-eat-a-low-oxalate-diet/, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress (see (Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906). In some embodiments, the beverage is a “Green smoothie” beverage comprising spinach, peanut butter, and soy milk. In some compositions, GM-ACT leaves are added to tea leaves. GM-ACT leaves may be provided in whole form or in ground form.

Methods of Extracting Oxalate from a Biological Sample

Methods for extracting oxalate from a biological sample are provided. Some methods are acid-base suspension (ABS) methods. Some methods include the steps of solubilizing oxalate with acid, removing unwanted material, precipitating the oxalate with ammonium hydroxide after the acid digestion step, removing unwanted solutes, and dissolving the oxalate pellet. In some methods ammonium hydroxide is used to wash the oxalate pellet after precipitation. In some methods, the oxalate pellet is dissolved in EDTA for analysis. In some embodiments, oxalate is extracted from a plant material. In some embodiments, the plant material is tea leaves, GM-ACT leaves, seagrass, or spinach. In some embodiments, the biological sample is a kidney stone. In some embodiments, the kidney stone is from a human. Acid-base suspension methods of the invention are easier and cheaper than prior methods which require use of high-performance liquid chromatography to separate the oxalate after dissolving the stone in acid (e.g., Litholink 24-Hour Urine Testing Kit, Litholink/Labcorp, Itasca, IL). Oxalate extracted from a biological sample by acid-base suspension methods of the invention may be detected in assays described herein using oxalate oxidase purified from GM-ACT leaves or for example using a commercial Trinity Biotech Oxalate Kit, (Cat. 591D, Bray, Co. Wicklow, Ireland).

Methods of Purifying Oxalate Oxidase from a Plant Material

Methods of purifying oxalate oxidase are provided including a step of extracting OxOx from plant leaves using 0.1% SDS that is heated to 60° C. In some embodiments, oxalate oxidase is purified from GM-ACT leaves. Oxalate oxidase purified from GM-ACT leaves using methods of the invention is useful as a commercial source for oxalate oxidase and source for oxalate oxidase as a stone disease therapeutic.

Diagnostic Uses and Methods of Detecting Oxalate

Oxalate oxidase purified from GM-ACT leaves may be used to detect and/or measure oxalate in a sample. In some embodiments, the sample is a biological sample. In some embodiments, the biological sample is from a human or animal. In other embodiments, the biological sample is from a plant. In some embodiments, the sample is from a food or beverage. In some embodiments, the sample is from a food or beverage, for example, beer, spinach, rhubarb, okra, peanut butter, taro, bran flakes, soy, tofu, soymilk, nuts (for example almonds, cashews, peanut), potatoes (including potato chips and French fries), beets, navy beans, raspberries, pumpkin, chocolate, swiss chard, eggplants, yams, avocados, tomato sauce, dates, and those reported at www.kidneystones.uchicago.edu./how-to-eat-a-low-oxalate-diet/, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress (see (Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906). In some embodiments, the sample is a “Green smoothie” beverage comprising spinach, peanut butter, and soy milk. In some embodiments, the plant is a tree. In some embodiments, the sample is wood. In some embodiments, the biological sample is from a tree infected with a tree pathogen. In some embodiments, the tree pathogen produces oxalate.

Oxalate oxidase purified from GM-ACT leaves may be used in a diagnostic assay and/or kit to measure oxalate in a biological sample, for example in urine or kidney stones. For example, oxalate oxidase purified from GM-ACT leaves may be used in a modification of the commercial Trinity Biotech Oxalate Kit, (Cat. 591D, Bray, Co. Wicklow, Ireland), replacing the kit-provided barley oxalate oxidase with oxalate oxidase purified from GM-ACT leaves.

Methods of Reducing Oxalate in a Material

GM-ACT leaves and/or oxalate oxidase purified from GM-ACT leaves may be added to a material to reduce oxalate levels. In some embodiments, the material is a food or beverage. In some embodiments, the material is a plant. In some embodiments, the plant is a tree. In some embodiments, the material is wood. The invention provides methods of reducing oxalate in foods and beverages using GM-ACT leaves and/or oxalate oxidase purified from GM-ACT leaves. Exemplary foods or beverages with high oxalate levels include beer, spinach, rhubarb, okra, peanut butter, tam, bran flakes, soy, tofu, soymilk, nuts (for example almonds, cashews, peanut), potatoes (including potato chips and French fries), beets, navy beans, raspberries, pumpkin, chocolate, swiss chard, eggplants, yams, avocados, tomato sauce, dates, and those reported at www.kidneystones.uchicago.edu./how-to-eat-a-low-oxalate-diet/, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress (see (Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906).

In some methods, the beverage is brewed from leaves of leaves of the Camellia sinensis shrub. In some methods, GM-ACT leaves are mixed with tea leaves. The GM-ACT leaves may be combined with the tea leaves in a package before brewing. Some packages are teabags.

GM-ACT leaves or oxalate oxidase purified from GM-ACT leaves may be used in methods to lower of the concentration of oxalic acid and in particular the prevention of the formation of calcium oxalate incrust and/or the degrading of precipitated calcium oxalate in the production of pulp and paper from wood.

Indications, Symptoms, and Disease Indicators

Many methods are available to assess development or progression of an oxalate-related disorder or a condition associated with elevated oxalate levels. Such disorders include, but are not limited to, any condition, disease, or disorder as defined above. Development or progression of an oxalate-related disorder may be assessed by measurement of urinary oxalate, plasma oxalate, measurement of kidney or liver function, or detection of calcium oxalate deposits, for example.

A condition, disease, or disorder may be identified by detecting or measuring oxalate concentrations, for example in a urine sample or other biological sample or fluid. An early symptom of hyperoxaluria is typically kidney stones, which may be associated with severe or sudden abdominal or flank pain, blood in the urine, frequent urges to urinate, pain when urinating, or fever and chills. Kidney stones may be symptomatic or asymptomatic, and may be visualized, for example by imaging the abdomen by x-ray, ultrasound, or computerized tomography (CT) scan. If hyperoxaluria is not controlled, the kidneys are damaged and kidney function is impaired. Kidneys may even fail. Kidney failure (and poor kidney function) may be identified by a decrease in or no urine output (glomerular filtration rate), general ill feeling, tiredness, and marked fatigue, nausea, vomiting, anemia, and/or failure to develop and grow normally in young children. Calcium oxalate deposits in other tissues and organs may also be detected by methods including direct visualization (e.g. in the eyes), x-ray, ultrasound, CT, echocardiogram, or biopsy (e.g. bone, liver, or kidney). Kidney and liver function, as well as oxalate concentrations, may also be assessed using well known direct and indirect assays. The chemical content or urine, blood or other biological sample may also be tested by well-known techniques. For example, oxalate, glycolate, and glycerate levels may be measured. Assays for liver and kidney function are well known, such as, for example, the analysis of liver tissue for enzyme deficiencies and the analysis of kidney tissue for oxalate deposits. Samples may also be tested for DNA changes known to cause primary hyperoxaluria.

Other indications for treatment and include, but are not limited to, the presence of one or more risk factors, including those discussed previously and in the following sections. A subject at risk for developing or susceptible to a condition, disease, or disorder or a subject who may be particularly receptive to treatment with oxalate oxidase may be identified by ascertaining the presence or absence of one or more such risk factors, diagnostic, or prognostic indicators. Similarly, an individual at risk for developing an oxalate-related disorder may be identified by analysis of one or more genetic or phenotypic markers.

Therapeutic Uses

Compositions and methods of the invention are useful in treating or reducing symptoms of oxalate-related disorders. Such diseases or conditions include, but are not limited to stone disease, hyperoxaluria, primarily hyperoxaluria, enteric hyperoxaluria, idiopathic hyperoxaluria, ethylene glycol (oxalate) poisoning, idiopathic urinary stone disease, renal failure (including progressive, chronic, or end-stage renal failure), steatorrhea, malabsorption, ileal disease, vulvodynia, cardiac conductance disorders, inflammatory bowel disease, cystic fibrosis, exocrine pancreatic insufficiency, Crohn's disease, ulcerative colitis, nephrocalcinosis, urolithiasis, breast cancer, nephrolithiasis, secondary hyperoxaluria (SH), Zellweger spectrum disorders (ZSD), autism, oxalosis associated with end-stage renal disease, colitis, sarcoidosis, asthma, COPD, fibromyalgia, Zellweger syndrome, bariatric surgery and other enteric disease states. Such conditions and disorders may optionally be acute or chronic. Oxalate-related disorders associated with kidneys, bone, liver, gastrointestinal tract, and pancreas are well known. Further, it is well known that calcium oxalate can deposit in a wide variety of tissues including, but not limited to the eyes, blood vessels, joints, bones, muscles, heart and other major organs leading to a number of oxalate-related disorders. Compositions and methods of the invention are also useful in treating breast cancer since it produces large amounts of oxalate.

GM-ACT leaves, alone or in combination with a food or beverage may be administered to a patient. Some foods or beverages that may be administered with GM-ACT leaves are beer, spinach, rhubarb, okra, peanut butter, taro, bran flakes, soy, tofu, soymilk, nuts (for example almonds, cashews, peanut), potatoes (including potato chips and French fries), beets, navy beans, raspberries, pumpkin, chocolate, swiss chard, eggplants, yams, avocados, tomato sauce, dates, and those reported at www.kidneystones.uchicago.edu./how-to-eat-a-low-oxalate-diet/, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress (see (Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906). In some methods, GM-ACT leaves are administered with tea leaves. In some methods, the GM-ACT leaves and tea leaves are brewed to produce a beverage for human consumption. In some methods, a tea prepared by methods of the invention is administered to a patient to reduce dietary oxalate load in the patient. In some methods, administration of the tea reduces and/or mitigates the patient's stone risk. In some methods, GM-ACT leaves alone, or in combination with other tea leaves, may mitigate the stone risk in stone formers who wish to enjoy the health benefits of freshly brewed tea.

In some embodiments, GM-ACT leaves are a prescription supplement that doctors, for example, urologists, prescribe to patients with an oxalate-related disorder or a condition associated with elevated oxalate levels, to be taken before a meal to degrade the oxalate during digestion. In some embodiments, the patient has stone disease, hyperoxaluria, and/or breast cancer. By reducing oxalate in the gut, oxalate is actively transported out of the blood into the gut. This leads to further reduction of urinary oxalate hence reducing stone disease recurrence which might be beneficial to high protein intake individuals (for example meat eaters and protein shake users). Once a person has a stone, 50 to 75% of individuals will have another stone within 5 to 10 years.

Consumption of meat leads to endogenous oxalate production. In some embodiments, GM-ACT leaves may be administered to a patient to reduce endogenous oxalate production that is associated with consumption of meat.

In addition, all aforementioned embodiments are applicable to domesticated, agricultural, or zoo-maintained animals suffering from an oxalate-related disorder, as well as to humans. For example, GM-ACT leaves or oxalate oxidase purified from GM-ACT leaves may be administered to house pets such as dogs, cats, rabbits, ferrets, guinea pigs, hamsters and gerbils, as well as to agricultural animals, such as horses, sheep, cows, poultry (e.g., chickens) and pigs.

Kits

The invention further provides kits comprising GM-ACT leaves or oxalate oxidase purified from GM-ACT leaves disclosed herein and related materials, such as instructions for use (e.g., package insert). The instructions for use may contain, for example, instructions for administration of the GM-ACT leaves or oxalate oxidase purified from GM-ACT leaves and optionally one or more additional agents. The kits can be sold for example, as a therapeutic agent for patients with an oxalate-related disorder or a condition associated with elevated oxalate levels. The instructions for use may contain, for example, instructions for using the oxalate oxidase purified from GM-ACT leaves in a diagnostic assay or a research assay for oxalate in a biological sample, and optionally one or more additional agents. The kits can be sold, for example, as research or diagnostic reagents.

Package insert refers to instructions customarily included in commercial packages of therapeutic products that contain information about the indications, usage, dosage, administration, contraindications and/or warnings concerning the use of such therapeutic products.

Kits can also include other materials desirable from a commercial and user standpoint, including other buffers and/or diluents and/or standards.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Examples

### Example 1:Acid-Base Resuspension Method

Methods: To access oxalate content in different substrates such as tea leaves, chestnut leaves, and seagrass, we developed a method by which we solubilize all the oxalate, remove unwanted material, precipitate the oxalate, remove unwanted solutes, and dissolve. In this method, ammonium hydroxide is used to precipitate after the acid digestion step and wash the oxalate pellet. EDTA to dissolve the oxalate pellet for analysis.

Up to 0.5 mg of leaf is incubated at 60° C. with 5M HCL for no less than 4 hrs. After 4 hrs, centrifuge at 10000 g for 10 min at 4° C. Collect supernatant and discard the pellet. Add 4 ml of 12M ammonium hydroxide (NH₄OH) and centrifuge at 10000 g for 10 min at 4° C. Discard supernatant and collect the pellet. Wash the pellet with 5M ammonium hydroxide and centrifuge at 10000 g for 10 min at 4° C. (this step can be repeated to clean the oxalate pellet). Resuspend the pellet in 1.0 to 0.5M EDTA (try to use the minimal volume needed to avoid diluting the oxalate). The resuspended oxalate can now be assayed with the Trinity assay.

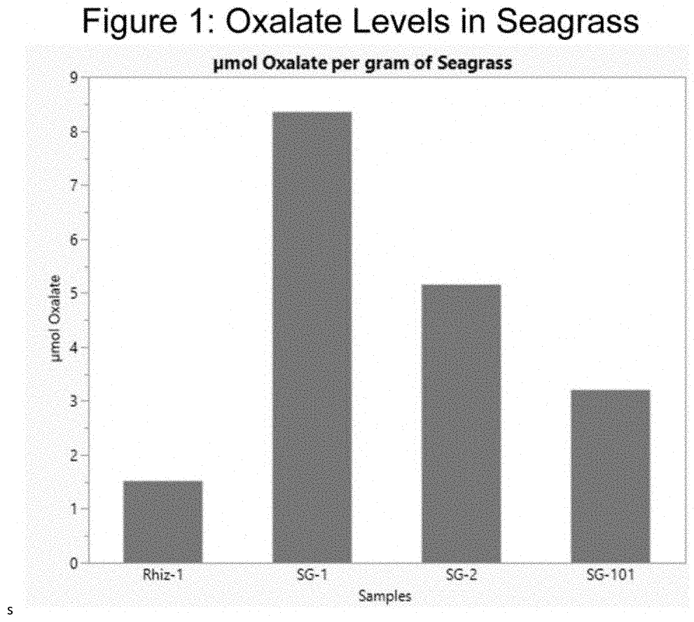

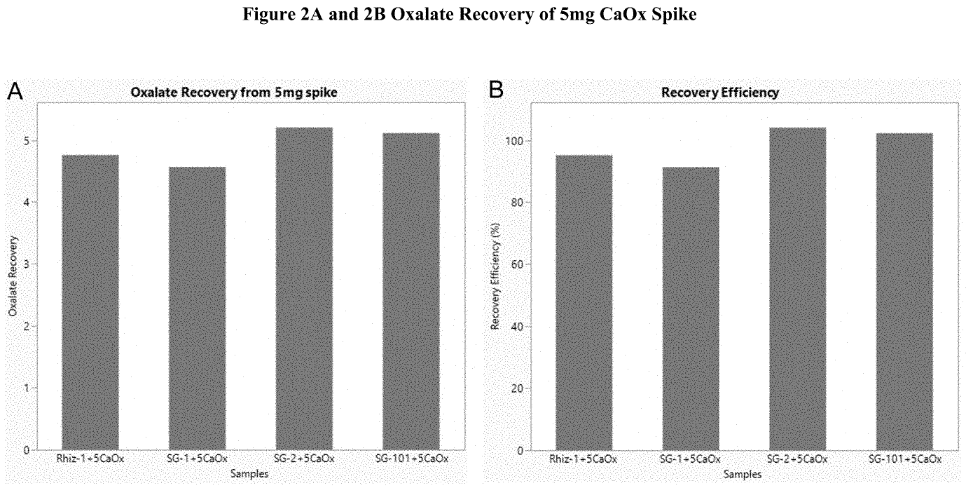

Measuring Oxalate in Leaves: In order to measure oxalate content in leaves, we developed a method utilizing the acid extraction method. But rather than neutralizing the acid to pH 7 which takes time, requires measuring the pH in the samples, and runs the risk of diluting the sample, we make the sample basic (˜pH14) by adding 12M NH₄OH. Afterwards, we recover the precipitate and resuspend it in EDTA ranging from 1.0 to 0.5M. FIG. 1 demonstrates oxalate levels in seagrass that was collected from different sites. To determine the efficiency of our recovery, the seagrass was spiked with 5 mg of calcium oxalate (FIG. 2A-2B).

### Example 2: Protocol for Quantifying Total Oxalate in Tea

This protocol starts with samples that have been dried and ground. This protocol is for quantifying total oxalate (soluble and insoluble combined) in dried, finely ground tissues.
1. Label two 15 ml conical tubes with each sample code
2. Homogenize the sample
   If in whirl packs, open the bag to let in air, close it, and carefully rotate a few times until the sample is mixed
3. Scoop sample into the conical tube and weigh it in a microscale until you have 0.5 g of sample
4. Record the exact weight on a data sheet (Excel)
5. Add 5 mL of 5M HCl (hydrochloric acid) to each sample
6. Vortex for 15 seconds
7. Put in the shaker incubator at 250 rpm for 4 hours at 60° C.
   a. It is OK to set to shake for 4 hours and hold at 60° C. until ready (overnight)
8. Centrifuge at 3500 rpm for 15 minutes at 20° C.
9. Pipette MOST (all the suspended liquid) of the supernatant to a new tube (labeled)
10. Take an aliquot (1 mL) of the supernatant, then store the supernatant in the freezer, labeled
11. Using the aliquot, supersaturate with calcium by adding 10% of the liquid volume of 6.75M CaCl₂) (calcium chloride)
   a. If there's 1 mL of liquid in the tube, add 100 μl of CaCl₂)
12. Vortex for 15 seconds
13. Add around 800-900 ul of ammonium hydroxide to each sample (record how much you used)
14. Vortex for 15 seconds
15. Centrifuge at 10,000 rpm for 15 minutes at 10° C.
16. Remove the supernatant using an aspirator or pipette. Do not disturb the pellet.
17. Add 100 μl EDTA ˜1M-0.5M
   a. Note: Add EDTA until the pellet is fully dissolved.
   b. Note: Usually around 50-100 ul is enough
18. Vortex for 15 seconds
19. Store in the fridge until ready to prepare dilutions or test
20. Centrifuge just bringing up to speed (no set time) to spin down any particles
21. Prepare a 96-well assay with original concentrations
   a. Each sample is tested in duplicate
   b. Each well gets 5 μl of sample using a P10 pipette
   c. A1 and A2 get 5 μl of 0.5 mM Oxalate (Standard)
   d. B1 and B2 get 5 μl of H₂O (Blank)
   e. C1 and C2 get 5 μl of sample 1, etc.
   f. Record the order so you can match readings to the sample code
22. Place plate in the Synergy microplate reader with a dual injector system for adding reagents
   a. Set to 37° C.
   b. Add 100 μl Reagent A=buffer and dye to make the sample reach the right pH
   c. Add 10 μl Reagent B=enzyme to start the reaction
   d. 4.5 min wait
   e. 0.5 min shake
   f. Read absorbency at 590 nm
23. The computer will give absorbency measurements for each sample. An acceptable measurement will be in between absorbency for H₂O and 0.5 mM Oxalate. Make predictions for any dilutions
24. SAVE THE EXCEL FILE TO A FLASH DRIVE WITH A DESCRIPTION
25. Prepare any samples that need to be diluted, using new microcentrifuge tubes (labeled)
   a. 2×=20 μl sample+20 μl H₂O
   b. 4×=10+30
   c. 5×=10+40
   d. 6×=10+50
   e. 7×=10+60
   f. 10×=10+90
   g. 15×=10+140
   h. 20×=10+190
   i. 25×=10+240
   j. 30×=5+145
   k. 50×=5+245
   l. 75×=5+370
   m. 100×=5+495
   n. 150×=5+745
   o. 200×=5+995
   p. 300×=5+1495
   q. 400×=3.75+1496.25
   r. 500×=3+1497
26. Repeat steps 21-25 until all samples are read in the appropriate range
27. Record the final dilution for each sample. If you did a 2× dilution on a sample that had a 5× dilution, then the final reading was a 10× dilution (2×5=10)
28. Copy the absorbency readings for each assay into the Trinity Biotech Oxalate Assay Excel sheet.
   a. There will be an Excel sheet for each assay run since the Blank and Standard are unique for each run and used in calculating oxalate for each sample.
   b. Make sure that “Mean-Blank” for the Blank is 0
   c. Enter the sample codes, final dilutions, and original mass (˜0.5 g—give exact) for each sample
29. Copy final measurement (mg Ox/g tissue) to an Excel sheet with all the samples together.
30. Clean up
   a. Shut down the Synergy microplate reader
   b. Samples are labeled with tape (initials, date, test details) and stored in the freezer
   c. Properly dispose of any waste

### Example 3: Protocol for Quantifying Soluble Oxalate in Tea

1. Weigh 2 g of ground samples into labeled conical tubes
2. Add 200 ml of boiling water and steep for 15 min
3. Vortex/shake for 15 seconds
4. Centrifuge at 2500 rpm for 15 min at 20° C.
5. Pipette the supernatant to new labeled tubes. This supernatant is ready to be tested. Start with step 21 of the total oxalate protocol.

Incubator max=120

Centrifuge max=48 15 ml tubes

### Example 4: Extraction of OxOx from American Chestnut Leaves

Methods: OxOx was extracted from GM-ACT leaves using 0.1% SDS that was heated to 60° C. The SDS and heat is needed to free OxOx from the cell wall. Previous patent (5,776,701; 07/07/1998) describes a method for extracting OxOx from barley seedlings. We differ in the use in that we use SDS and it is heated at the time of blending the chestnut leaves rather than post processing.

40 g of GM-ACT leaves were blended using 60° C. 0.1% SDS for 5 min. Enough SDS is added till a slurry is achieved. The Slurry is passed through a masticating juicer 3 times to break the cell walls for better extraction. The resulting GM-ACT juice (fiber is discarded) is then centrifuged at 10000 g to remove remaining fibers. Adjust Chestnut supernatant to contain 30% w/v Ammonium Sulfate and centrifuge at 10,000 g for 10 min at 20° C. Discard pellets and keep supernatant. Adjust Chestnut supernatant to contain 70% Ammonium Sulfate. Centrifuge at 10,000 g for 60 min at 10° C. Discard supernatant and keep the pellet this time. Resuspend pellets in DI water till it's mostly dissolved. Dialysis overnight at room temperature. Dialyzed OxOx was evaluated using a modified Trinity Assay protocol to measure enzymatic activity.

Results:

The use of 0.1% SDS at 60° C. during the blending process improves yield of nearly 10-fold improvement of enzymes per 100 g of leaves (Table 1). The reported yield of barley is approximately 1 unit per 100 g seedlings. The GM-ACT leaves OxOx yield is nearly 10-fold greater.

TABLE 1
Oxalate Oxidase Yield in GM-ACT Leaves

| | GM-ACT | GM-ACT with |
|---|---|---|
| GM-ACT | with DI water | 60° C. 0.1% |
| with DI water | at 60° C. | SDS |
| 1.08 units per | 4.84 units per | 9.74 units per |
| 100 g leaf | 100 g leaf | 100 g leaf |

### Example 5:OxOx Extraction from GM-ACT Leaves

1. Prepare 0.1% SDS solution
   a. Use DI water
   b. Adjust pH to approximately pH ˜4
2. Heat SDS solution to 60° C. before weighing Chestnut leaves.
   a. Recommend warming of blender carafe to 60° C. as well.
3. Weigh no less than 20 g of leaves
   a. Lower than 20 g greatly affects extraction efficiency (More is better)
   b. Recommended 40 g or greater
   c. Weigh leaves in a cold room or using a chilled container to minimize frozen leaves turning into mush.
4. Blend the leaves
   a. Add 40 g of leaves and 10 ml of 60° C. SDS solution
   b. Add more 60° C. SDS solutions till a slurry is achieved
   c. We used 30 yr Oster blend set to liquefy
5. Juice the Slurry
   a. Transfer the slurry slowly into a juicer
   b. Collect the pulp and pass twice more through the juicer
   c. Take the pulp and add it back into the chestnut leaf juice.
      i. Alternative: Wet pulp with SDS solution; however, will end up with a larger final volume to salt out the OxOx
   d. Pass this through the juicer
   e. Collect and pass the pulp 2× again.
   f. Keep the chestnut juice and discard the pulp.
6. Ammonium Sulfate Precipitation of OxOx
   a. Adjust Chestnut Juice to contain 30% Ammonium Sulfate.
   b. Centrifuge at 10,000 g for 10 min at 20° C.
   c. Discard pellet and keep supernatant
   d. Adjust Chestnut Juice to contain 70% Ammonium Sulfate
      i. www.encorbio.com/protocols/AM-SO4.htm
   e. Centrifuge at 10,000 g for 60 min at 10° C.
      i. Note: remember to change temp to 10° C. from 20° C.
   f. Discard supernatant and keep the pellet this time.
      i. Note: Do not discard the pellet
   g. Resuspend pellets in DI water till they are mostly dissolved.
      i. Expect some material not to dissolve such as tar
7. Dialysis
   a. Dialysis overnight in a cold room or fridge.
      i. For a 25 ml volume, place the dialysis tube in 4 L of DI water with a stir rod.
      ii. After 1 to 2 h, replace the DI water bath.
         1. Repeat this step at least twice
         2. This helps remove dyes that can affect OxOx colorimetric assay
      iii. Place in cold room for overnight dialysis
         1. Alternatively, overnight at room temp appears to have no significant effect
   b. After dialysis, centrifuge any particles.
   c. The neat solution is ready for testing.

### Example 6: Comparison of GM-ACT Leaf Extract with Trinity Biotech Oxalate Oxidase

An extract from GM-ACT leaves was compared with oxalate oxidase provided in a modified Trinity Biotech Oxalate assay (Reagent B).

Methods:
1. Mix:

5 μL of 0.5 mM oxalate standard or H₂O₂ Standards +10 μL of GM-ACT extract prepared as in Example 5 or Trinity Biotech Reagent B +100 μL Trinity Biotech Reagent A.
2. Incubate 5 minutes at 37° C.
3. Read absorbency at 590 nM.

Results: The American Chestnut leaf extract has the equivalent performance of 1/10 dilution Trinity Biotech oxalate oxidase.

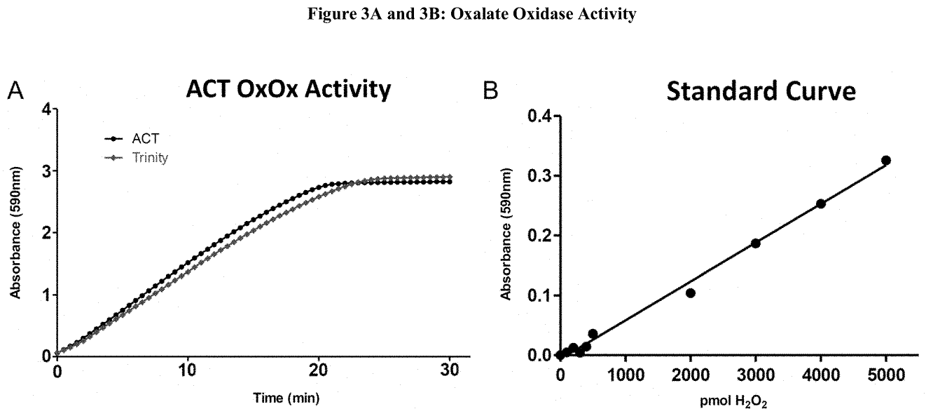

FIG. 3A shows a comparison of GM-ACT leaf extract to 1/10 dilution of oxalate oxidase enzyme from the Trinity Biotech oxalate assay. FIG. 3B: Hydrogen peroxide (byproduct of oxalate degradation) standard curve for determination of activity.

FIG. 3A shows that GM-ACT is useful as a commercial source for oxalate oxidase and a source for oxalate oxidase as a stone disease therapeutic.

### Example 7: Preparation of Dried and Ground GM-ACT Leaves

Leaves were dried at 60° C. at least overnight but not greater than 3 days.
Leaves were ground using a Krups model F203 coffee grinder for 30 seconds to 60 seconds.
Powder was passed through a strainer to remove any large particles such as stem remains.

### Example 8:Oxalate Reduction by American Chestnut Leaves

Methods: To determine the activity of the GM-ACT leaves, a titration of 5 ml volumes of sodium oxalate (NaOx) was used at the following concentrations: 0.1, 1, 10, 10, and 100 mM. The 5 ml of NaOx was added to 50 mg of ground GM-ACT leaves. The reactions were observed at 30, 60, 120, 180, 300, and 600 s. Reaction conditions were held at 37° C. and shaken at 250 rpms. All reactions occurred in 50 ml conical tubes.

American Chestnut Leaves Degrade Oxalate: When determining the oxalate reduction of GM-ACT leaves, we looked at the ability of the leaf ground itself vs. the tea made from the leaves. Even after exposure to hot water, the ground leaves were capable of degrading oxalate; however, the tea had no activity. This is due to OxOx being tightly bound in the cell walls of the leaf. Without SDS, OxOx remains in the ground GM-ACT leaves. The ground GM-ACT leaves are acting as microparticles with high efficiency. The enzyme activity units per gram GM-ACT leaves were 255 units/g. In other words, for 100 grams of leaves, this amount would be equivalent to 25,500 units which is substantially higher than the 1 unit/g obtained from barley.

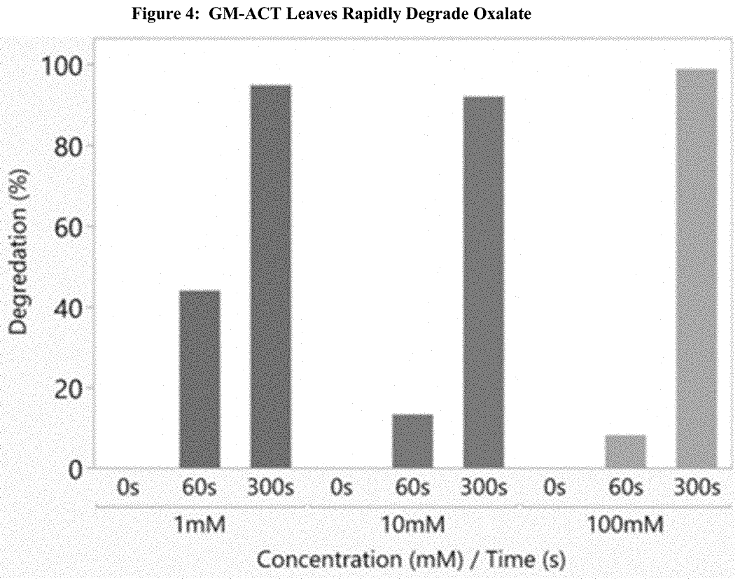

FIG. 4 demonstrates the ability of ground chestnut leaves to degrade oxalate in a very short time. 50 mg of GM-ACT leaf can degrade up to 64 mg (500 μmol) of oxalate in 5 min. Stone patients that are high absorbers can excrete up to 66 mg of oxalate after consuming 8 oz of spinach. An excretion of 40 mg or greater is considered hyperoxaluric indicating a kidney stone former. Potentially 50 mg of GM-ACT leaves could mitigate 97% of the oxalate.

### Example 9: Reduction of Oxalate in Tea by American Chestnut Leaves

Methods: A total of 6 teas were brewed per the manufacturer's recommendations (Yorkshire, Lipton, Tazo, PG Tips, Twinings, and Greenwise) using calcium/magnesium-free phosphate buffered saline (PBS). Distilled and spring water was also used with Lipton teas to assess the difference in oxalate reduction between the brewing solutions.

To assess oxalate reduction, we first measured the amount of oxalate content in 250 mg of tea leaves without GM-ACT leaves. Free leaves of Yorkshire, Lipton, Tazo, PG Tips, Twinings, and Greenwise teas were brewed at manufacturer's recommended serving size in 25 ml of boiling Ca+/Mg+ free phosphate buffered saline (PBS)×5 minutes. Oxalate content was measured by colorimetric assay (Trinity Biotech) in triplicate. Then, 250 mg of tea with 250 mg of GM-ACT leaves were steeped in 25 ml of boiling PBS and/or water for 5 min. Oxalate was measured using the colorimetric Trinity Biotech Oxalate Assay in triplicate. Lipton tea (250 mg) was steeped (5′) in 25 ml of distilled and spring water to assess the effect of solution on oxalate content.

To analyze the data, we calculated the mean milligrams of soluble Oxalate (mg Ox) per sample of 0.25 g of each tea's leaves (“tissue”), both with and without the additional 0.25 mg GM-ACT leaves. Doing so allowed us to assess the potency of each tea at a standardized level. However, for a more practical analysis of the data, we also calculated the mean mg Ox per serving size (varies according to the manufacturer), with and without the additional 0.25 mg GM-ACT leaves. The latter allows for the assessment of oxalate consumption when ingesting the tea as directed on each tea's box.

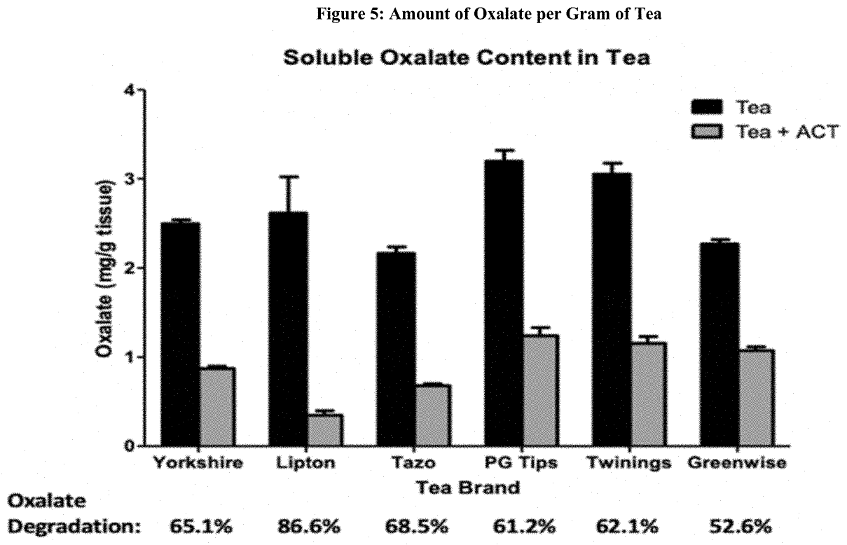

American Chestnut Leaves Reduce Oxalate in Tea: When viewing each tea, the consistent theme was that oxalate levels were considerably lower after steeping the tea leaves with GM-ACT leaves. We found that before adding GM-ACT leaves, there was a range of 2.16 to 3.19 mg Ox per 0.25 g of tea leaves (FIG. 5, with Tazo being the lowest amount of soluble oxalate per gram of GM-ACT leaves, and PG Tips being the highest. When GM-ACT leaves were steeped with the tea, soluble oxalate was reduced by an average of 66.01%, with Greenwise being the lowest reduction at 52.6% and Lipton having the highest reduction at 86.6%.

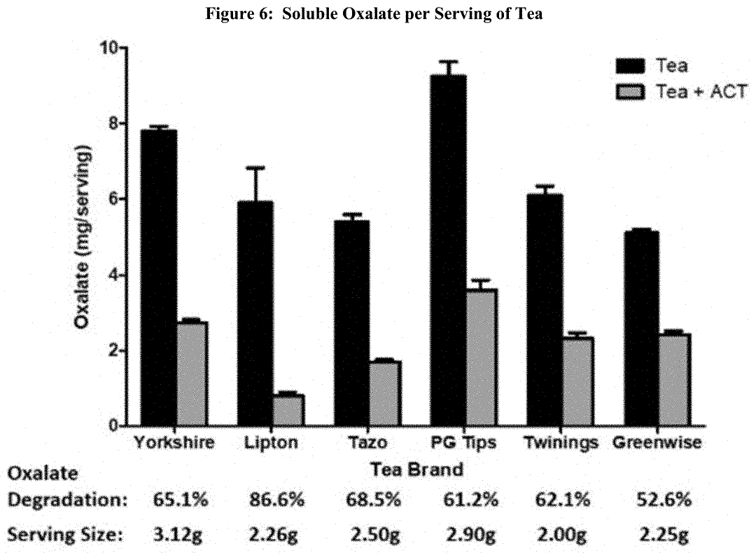

When viewing the data in terms of mg Ox per serving sizes of the tea leaves, we see a similar trend once the GM-ACT leaves are added (FIG. 6). We found that before adding GM-ACT leaves, there was a range of 5.1 to 9.3 mgOx per serving size, with PG Tips being the highest and Greenwise being the lowest amount of soluble oxalate per serving. Again, this view of the data allows us to analyze how much soluble oxalate will be ingested if the tea is consumed per the brand's direction.

Results:

FIG. 5: We consistently found that leaf “tissue” oxalate levels were considerably lower after adding OxOx leaves. Before adding GM-ACT leaves, soluble oxalate ranged from 2.16 to 3.19 mg Ox per g of tea leaves. When GM-ACT leaves were steeped with tea, soluble oxalate was reduced, on average, of 66.02%, with the individual variations shown and largest variation was observed in Lipton tea.

FIG. 6: In terms of mg Ox per serving sizes of tea leaves, a similar trend as in FIG. 1 is noted when GM-ACT leaves are added. Prior to adding GM-ACT leaves, soluble oxalate ranged from 5.1 to 9.3 mg Ox per serving size, with the individual variations shown.

When Lipton black tea steep solution varied, we saw no statistically significant difference in soluble oxalate levels. PBS had slightly higher soluble oxalate compared to distilled (0.672 mg Ox/gm of tissue) or spring water (0.732 mg Ox/gm of tissue), indicating that brewing solution does not affect soluble oxalate content.

### Example 10: Assay for Reduction of Oxalate in Tea by GM-ACT Leaves

Amounts & Volumes
Teas
   Triplicates for each experiment
   0.25 g
GM-ACT Leaves
   Triplicates for each experiment
   0.25 g
PBS without Ca/Mg
   25 ml

Procedure
1. Tea (0.25 g) with/without GM-ACT leaves (0.25 g) will be steeped in 25 ml of boiling PBS
   5 min
   Shaker Incubator 250 RPM
2. After 15 min, Centrifuge
   5000 g @ 4° C.
   5 min
   Discard pellet & Keep the Tea
3. Aliquot tea (See LABELING Aliquots section)
   1-2 ml aliquots in 2 ml microcentrifuge tubes
   Store in −20° C. or −80° C. Freezer
   At the point, experiment can stop and continued at a later time
4. Test using Trinity Assay

LABELING Aliquots
Teas in triplicate
   X
      X for whatever tea name or designation you use
      X1-X3
GM-ACT Leaf
   X in triplicate
      X4-X6

### REFERENCES

1 Rule A D, Krambeck A E, Lieske J C. Chronic kidney disease in kidney stone formers. Clin J Am Soc Nephrol. 2011 August; 6(8):2069-75.
2. Dominguez-Gutierrez P R, Kusmartsev S, Canales B K, Khan S R. Calcium oxalate differentiates human monocytes into inflammatory M1 macrophages. Front Immunol. 2018 Aug. 22; 9:1863.
3. Khan S R, Canales B K, Dominguez-Gutierrez P R. Randall's plaque and calcium oxalate stone formation: role for immunity and inflammation. Nat Rev Nephrol. 2021 Jun; 17(6):417-33.
4. Carlson E, Stewart K, Baier K, McGuigan L, Culpepper T, Powell W. Pathogen-induced expression of a blight tolerance transgene in American chestnut. Mol Plant Pathol. 2022 Mar; 23(3):370-82.
5. Powell W A, Newhouse A E, Coffey V. Developing Blight-Tolerant American Chestnut Trees. Cold Spring Harb Perspect Biol. 2019 Jul. 1; 11(7).
6. Strauss S H, Boerjan W, Chiang V, Costanza A, Coleman H, Davis J M, et al. Certification for gene-edited forests. Science. 2019 Aug. 23; 365(6455):767-8.
7. U.S. Pat. No. 5,776,701A—Materials and methods for detecting oxalate -
8. Holmes R P, Goodman H O, Assimos D G. Contribution of dietary oxalate to urinary oxalate excretion. Kidney Int. 2001 January; 59(1):270-6.
9. Holmes R P, Assimos D G. The impact of dietary oxalate on kidney stone formation. Urol Res. 2004 October; 32(5):311-6.
10. Khan S R, Glenton P A, Byer K J. Dietary oxalate and calcium oxalate nephrolithiasis. J Urol. 2007 November; 178(5):2191-6.
11. Robijn S, Hoppe B, Vervaet B A, D'Haese P C, Verhulst A. Hyperoxaluria: a gut-kidney axis? Kidney Int. 2011 December; 80(11):1146-58.
12. WCE 2018: Reduction of Urinary Oxalate by Enzymatic Reduction of Dietary and Endogenous Oxalate [Internet]. [cited 2019 Feb. 1]. Available from: www.urotoday.com/conference-highlights/wce-2018/106962-wce-2018-reduction-of-urinary-oxalate-by-enzymatic-reduction-of-dietary-and-endogenous-oxalate.html
13. Whittamore J M, Hatch M. The role of intestinal oxalate transport in hyperoxaluria and the formation of kidney stones in animals and man. Urolithiasis. 2017 February; 45(1):89-108.
14. Holmes R P, Knight J, Assimos D G. Lowering urinary oxalate excretion to decrease calcium oxalate stone disease. Urolithiasis. 2016 February; 44(1):27-32.
15. Anon Can Genetic Engineering Bring Back the American Chestnut?—The New York Times [Online]. Available at: www.nytimes.com/2020/04/30/magazine/american-chestnut.html
16. Taylor, E. N. and Curhan, G. C. 2007. Oxalate intake and the risk for nephrolithiasis. Journal of the American Society of Nephrology 18(7), pp. 2198-2204.
17. Hill A J, Basourakos S P, Lewiciki P, Wu X, Arenas-Gallo C, Chuang D, et al. Incidence of kidney stones in the united states, the continuous national health and nutrition examination survey. J Urol. 2021 Dec. 2.
18. Dratewka-Kos, E., Rahman, S., Grzelczak, Z. F., Kennedy, T. D., Murray, R. K., and Lane, B. G. (1989). Polypeptide structure of germin as deduced from cDNA sequencing. J. Biol. Chem. 264, 4896-4900.
19. Newhouse, A., (2020_FinalChestnut Winter2020, Safety tests on transgenic American Chestnut, part 1, The American Chestnut Foundation, Winter 2020, pg. 26-27.
20. Zhang, B. et al., (2013) A threshold level of oxalate oxidase transgene expression reduces Cryphonectria parasitica-induced necrosis in a transgenic American chestnut (Castanea dentata) leaf bioassay, Transgenic Res. 2:973-982.
21. Earnest, Adv. Internal Medicine 24:407-427 (1979).
22. Massey, L. K., H. Roman-Smith, and R. A. L. Sutton (1993) Effect of dietary oxalate and calcium on urinary oxalate and risk of formation of calcium oxalate kidney stones, Journal of the American Dietetic Association. 93: 901-906)
22. www.kidneystones.uchicago.edu./how-to-eat-a-low-oxalate-diet/
23. www.encorbio.com/protocols/AM-SO4.htm

What is claimed is:

1. A composition comprising tea leaves and leaves of American chestnut tree plants expressing wheat oxalate oxidase.

2. The composition of claim 1, wherein the leaves of American chestnut tree plants expressing wheat oxalate oxidase are ground.

3. A method for producing a brewed tea with reduced oxalate levels, comprising:
(a) combining tea leaves with leaves of American chestnut tree plants expressing wheat oxalate oxidase;
(b) steeping the tea leaves and leaves of American chestnut tree plants expressing wheat oxalate oxidase in boiling water to brew the tea; and
(c) collecting the brewed tea,
wherein the brewed tea has reduced oxalate levels compared to tea brewed without leaves of American chestnut tree plants expressing wheat oxalate oxidase.

4. The method of claim 3, wherein the leaves of American chestnut tree plants expressing wheat oxalate oxidase are ground.

5. A method of reducing stone formation in a patient, comprising administering a therapeutically effective amount of leaves of American chestnut tree plants expressing wheat oxalate oxidase to the patient, wherein stone formation is reduced in the patient.

6. A method of reducing urinary oxalate levels in a patient, comprising administering a therapeutically effective amount of leaves of American chestnut tree plants expressing wheat oxalate oxidase to the patient, wherein urinary oxalate levels are reduced in the patient.

7. A method for purifying oxalate oxidase from leaves of American chestnut tree plants expressing wheat oxalate oxidase, the method comprising:
preparing an extract from the leaves, wherein the extract is prepared in the presence of 0.1% sodium dodecyl sulfate at 60° C., precipitating proteins in said extract by bringing said extract to at least about 30% (w/v) saturation with ammonium sulfate, removing said precipitated proteins and bringing said extract to at least about 60% (w/v) saturation with ammonium sulfate to precipitate said oxalate oxidase present in said extract and recovering said oxalate oxidase.

8. An oxalate oxidase composition having a specific activity of at least about 9 enzyme units per 100 g of starting leaves of American chestnut tree plants expressing wheat oxalate oxidase after extraction in the presence of 0.1% sodium dodecyl sulfate at 60° C. and ammonium sulfate precipitation.

9. The oxalate oxidase composition according to claim 8 wherein said oxalate oxidase is produced according to a process comprising the steps of preparing an extract from leaves of American chestnut tree plants expressing wheat oxalate oxidase, wherein the extract is prepared in the presence of 0.1% sodium dodecyl sulfate at 60° C., precipitating proteins in said extract by bringing said extract to at least about 30% saturation (w/v) with ammonium sulfate, removing said precipitated proteins and bringing said extract to at least about 60% (w/v) saturation with ammonium sulfate to precipitate said oxalate oxidase present in said extract and recovering said oxalate oxidase.

10. A method of reducing oxalate levels in a material, comprising adding an effective amount of the oxalate oxidase composition of claim 8 to the material, wherein the oxalate level of the material is reduced.

11. The method of claim 10, wherein the material is a food or beverage.

12. The method of claim 11, wherein the food or beverage is selected from the group consisting of beer, spinach, rhubarb, okra, peanut butter, taro, bran flakes, soy, tofu, soymilk, nuts, almonds, cashews, peanuts, potatoes, beets, navy beans, raspberries, pumpkin, chocolate, swiss chard, eggplants, yams, avocados, tomato sauce, dates, strawberries, wheat bran, tea, leafy green vegetables, asparagus, runner beans, beetroot, Brussels sprouts, cabbage, carrots, cauliflower, celery, chives, lettuce, marrow, mushrooms, onions, parsley, green peas, radishes, tomatoes, turnips, apples, apricots, ripe bananas, gooseberries, grapefruits, melons, oranges, peaches, pears, pineapples, plums, blueberries, arugula, beet greens, collard greens, kale, endive, bok choy, dandelion greens, escarole, cole, mache, mustard greens, radicchio, rapini, and watercress.

13. The method of claim 10, wherein the material is wood.

14. A method for extracting oxalate from a biological sample, comprising:
(a) solubilizing oxalate in the biological sample with 5 M HCl at 60° C. for 4 hours;
(b) separating the solubilized oxalate from insoluble material;
(c) precipitating oxalate from the solubilized oxalate with 12 M ammonium hydroxide;
(d) washing the precipitated oxalate with 5 M ammonium hydroxide; and
(e) resuspending the washed precipitated oxalate in EDTA at a concentration of 0.5 M or 1.0 M.

15. The method of claim 14, wherein the biological sample is tea leaves, leaves of American chestnut tree plants expressing wheat oxalate oxidase, seagrass, spinach, or kidney stone.

16. The method of claim 14, wherein the kidney stone is from a human.
